(19) United States
(12) Patent Application Publication
(10) Pub. No.: US 2008/0072365 A1
(43) Pub. Date: Mar. 27, 2008

(54) Space-saving scuba diving mask

(75) Inventor: Carlos Alberto, Genova (IT)

Correspondence Address: POLLACK, P.C., THE CHRYSLER BUILDING, 132 EAST 43RD STREET, SUITE 760, NEW YORK, NY, 10017, US

(73) Assignee: Cressi-Sub S.p.A.

(21) Appl. No.: 11/901,382

(22) Filed: Sep. 16, 2007

(30) Foreign Application Priority Data
Sep. 15, 2006 (IT) FI2006U000057

(51) Int. Cl. A61F 9/02

(52) U.S. Cl. 2/428; 2/426; 2/452

(57) ABSTRACT

A scuba diving mask comprising a rigid frame carrying at least one transparent lens, and a flexible skirt connected to the perimeter of the frame. The mask also comprises a strap for holding the mask against the user's head and a pair of buckles for adjusting the length of the strap connected to the lateral sides of the frame by means of brackets extending therefrom, the brackets being made of a flexible material.

### FIELD OF THE INVENTION

The present invention relates generally to storage and, more particularly, to equipment for swimming and underwater activities.

### BACKGROUND OF THE INVENTION

Scuba diving masks typically comprise a rigid frame supporting a transparent lens, or “visor”, and a flexible gasket or “skirt” attached to the perimeter of the frame suitable for resting against the scuba diver's face so as to provide the necessary watertightness. A support strap, jointed to either side of the mask, is usually also provided to ensure a close fitting of the mask against the scuba diver's face.

The frame can also be made in two parts, in which case the visor consists of two symmetrical lens elements.

In masks of the known type, the two ends of the supporting strap are equipped with a buckle that allows for the adjustment of the strap's length so as to enable the mask to be adapted to the dimensions of the user's head.

The two buckles may be connected either to the two sides of the rigid frame or to the two sides of the flexible skirt of the mask.

In the former case, a pair of brackets lying substantially perpendicular to the plane of the visor and facing towards the rear of the mask extend rigidly from the mask frame. These two brackets are consequently aligned with the direction that the ends of the supporting strap tend to occupy when the mask is being worn. The two brackets are rigid as the frame of the mask is, thus resulting in the increase of the overall dimensions of the mask in the direction perpendicular to the plane of the visor. This configuration influences the costs involved in the transport of the mask (because the mask has to be placed in a container of appropriate dimensions), and makes some troubles to the user when the mask has to be stored.

In the case of the second known type of connection, the buckles on the straps are attached directly to the flexible skirt, so the problem of the mask overall dimensions when it has to be transported or stored would appear to have been overcome.

However, this second known type of solution has several drawbacks. As a matter of fact, this second solution has so far only been applied to a very limited number of models. The first drawback consists in that any strap-tightening action modifies the shape of the flexible skirt, with an unavoidable loss of watertightness when this action is taken underwater. A second drawback lies in that, when the mask is being adjusted, a tensile stress is produced on the flexible skirt referring to the point where the buckle is attached, thus resulting in the rupture of the skirt after a certain number of adjustment operations.

### OBJECTS AND SUMMARY OF THE INVENTION

The object of the present invention is to provide a scuba diving mask that allows the above drawbacks of scuba diving masks of the known type to be avoided.

A particular object of the present invention is to provide a scuba diving mask wherein the connection between the frame and the adjustment strap is such that it allows the overall dimensions of the mask to be reduced when the mask must be placed in a box for packaging and dispatch operations, or stored after use, without this entailing any risk of damage to the skirt after the repetition of a certain number of adjustment operations of the length of the strap.

These objects are achieved with a scuba diving mask according to the present invention, characterized in that the brackets connecting the frame to the strap adjustment buckles are made of a flexible material.

### BRIEF DESCRIPTION OF THE DRAWINGS

A specific, illustrative space-saving scuba diving mask, according to the present invention, is described below with reference to the accompanying drawings, in which:

FIG. 1 is a perspective view of a scuba diving mask, according to one aspect of the present invention;

FIG. 2 is a plan view of the mask shown in FIG. 1; and

FIG. 3 is a plan view of the mask illustrated in FIG. 1 in a folded position.

Still other objects and advantages of the present invention will become apparent from the following description of the preferred embodiments.

### DETAILED DESCRIPTION OF THE PREFERRED EMBODIMENTS

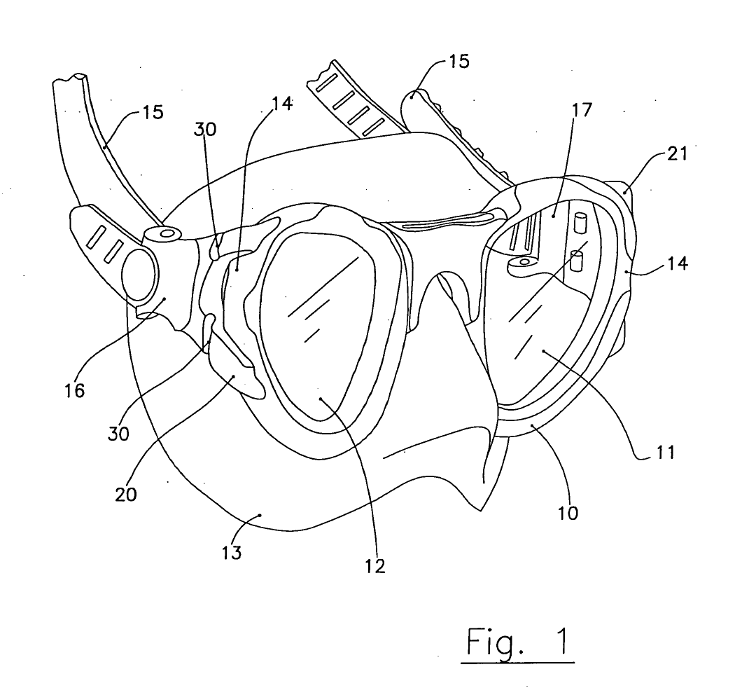

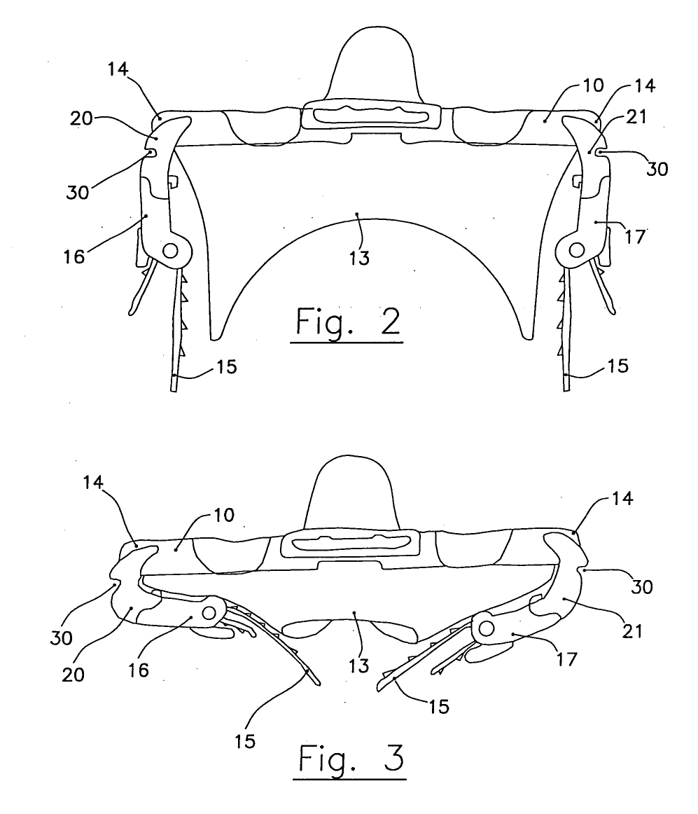

Referring now to the drawings and, more particularly, to FIGS. 1-4, there is shown generally a specific, illustrative space-saving mask, according to various aspects of the present invention. In one embodiment, Illustrated generally in FIG. 1, the mask has a relatively rigid frame 10 that, for instance, has a pair of visors or lenses, 11 and 12. A flexible skirt 13 is integrally attached to the perimeter of the frame by a watertight connection. The numeral 14 identifies the lateral sides of the frame and the numeral 15 identifies a flexible strap that has its two ends connected to two buckles 16 and 17, which allows its length to be adjusted.

According to the invention, the buckles 16 and 17 are attached to the lateral sides 14 of the frame by means of two brackets 20 and 21, made of a flexible material, that extend on a plane substantially perpendicular to the frame. The buckles of flexible material are molded directly onto the material of the frame and the two materials are connected together by means of a chemical bond. The materials involved may be a thermoplastic rubber and a polycarbonate, for instance. The two brackets 20 and 21 can be connected to the buckles 16 and 17 by coupling means of known type, such as pivots and the like (not shown).

The advantages of the invention are illustrated in FIG. 3, wherein the mask is shown in a top plan view with the buckles 16 and 17 positioned substantially against the frame of the mask thanks to the folding of the brackets 20 and 21. To make easier their folding, the two brackets are preferably provided with one or more grooves 30 that reduce their cross-section.

As a result, the overall dimensions of the mask are reduced to make easier its transport from the manufacturer to the retailer. The reduced dimensions can also be useful to the user, who can place the mask in a normal pocket or in a standard spectacle case.

Various modifications and alterations to the present invention maybe appreciated based on a review of this disclosure. These changes and additions are intended to be within the scope and spirit of the invention as defined in by the following claims.

What is claimed is:

1. A scuba diving mask comprising a rigid frame carrying at least one transparent lens, a flexible skirt connected to the perimeter of the frame, a strap for holding the mask against the user's head and a pair of buckles for adjusting the length of the strap connected to the lateral sides of the frame by brackets extending from the lateral sides of the frame, wherein the brackets are made of a flexible material.

2. The scuba diving mask set forth in claim 1, wherein the brackets are molded directly onto the rigid frame.

3. The scuba diving mask set forth in claim 1, wherein one or more grooves are formed on the brackets lying parallel to the plane of the at least one transparent lens to facilitate the folding of the brackets.

4. The scuba diving mask set forth in claim 1, wherein one or more grooves are formed on the brackets lying parallel to the plane of the at least one transparent lens to facilitate the folding of the brackets.
